(19) United States
(12) Patent Application Publication
(10) Pub. No.: US 2007/0173855 A1
(43) Pub. Date: Jul. 26, 2007

(54) Devices and methods for spacing of vertebral members over multiple levels

(75) Inventors: Brad Winn, Southaven, MS (US); Craig Squires, Cordova, TN (US); Mark C. Dace, Collierville, TN (US); Bret Wilfong, Southaven, MS (US); Jared Shoup, Memphis, TN (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/333,720

(22) Filed: Jan. 17, 2006

(51) Int. Cl. A61B 17/58

(52) U.S. Cl. 606/90

(57) ABSTRACT

The present application is directed to devices and methods to space apart vertebral members over two or more spinal levels. Embodiments of the devices may include a power source, supply line, and adjustable members. The adjustable members may be positioned along two or more spinal levels. The supply line may extend between each of the members and the power source. Activation of the power source may feed power through the supply line and to each of the adjustable members. Members may move from a closed orientation towards an open orientation to space the vertebral members.

### BACKGROUND

The present application is directed to device and methods for moving vertebral members, and more specifically, to devices and methods for spacing vertebral members over multiple levels.

The spine is divided into regions that include the cervical, thoracic, and lumbar regions. The cervical region includes the top seven vertebral members identified as C1-C7. The thoracic region includes the next twelve vertebral members identified as T1-T12. The lumbar region includes five vertebral members L1-L5. The vertebral members are spaced apart forming an intervertebral space between each adjacent vertebral member. Intervertebral discs are located within this space and permit slight flexion, extension, lateral flexion, and rotation.

Various procedures include spacing apart the vertebral members that extend along a section of the spine. These procedures may be required due to damage to one or more of the vertebral members and/or intervertebral discs. The damage may be caused by a specific event such as trauma, a degenerative condition, a tumor, or infection. Currently, decompression of vertebral members along a spinal section is completed independently at each spinal level. These techniques have the potential for applying too much force at one or more levels that could affect the single or multilevel kinematics of the adjacent spinal levels.

### SUMMARY

The present application is directed to devices and methods to space apart vertebral members over two or more spinal levels. One embodiment may include a power source, a supply line, and two or more expandable members. Each of the members may be placed at different locations along the spine. The supply line may operatively connect the members with the power supply. Activation of the power supply may cause each of the expandable members to increase in height and space apart the vertebral members within the spinal levels at issue.

### BRIEF DESCRIPTION OF THE DRAWINGS

FIG. 1 is a schematic diagram illustrating a device for spacing vertebral members according to one embodiment.

FIG. 2 is a perspective view of an expandable member according to one embodiment.

FIG. 3A is a schematic diagram of a member in a first orientation and positioned between vertebral members according to one embodiment.

FIG. 3B is a schematic diagram of a member in a second orientation and positioned between vertebral members according to one embodiment.

FIG. 4 is a perspective view of a member according to one embodiment.

FIG. 5 is a schematic diagram illustrating a device for spacing vertebral members according to one embodiment.

FIG. 6 is a flowchart diagram illustrating the steps of using a spacing device according to one embodiment.

### DETAILED DESCRIPTION

The present application is directed to devices and methods to space apart vertebral members over two or more spinal levels. The devices and methods may include placing expandable members within two or more levels of vertebral members. The expandable members may be connected by a supply line to a power source. Activation of the power source may feed power throughout the supply line and to two or more of the expandable members causing the members to increase in height and apply a common force to the vertebral members.

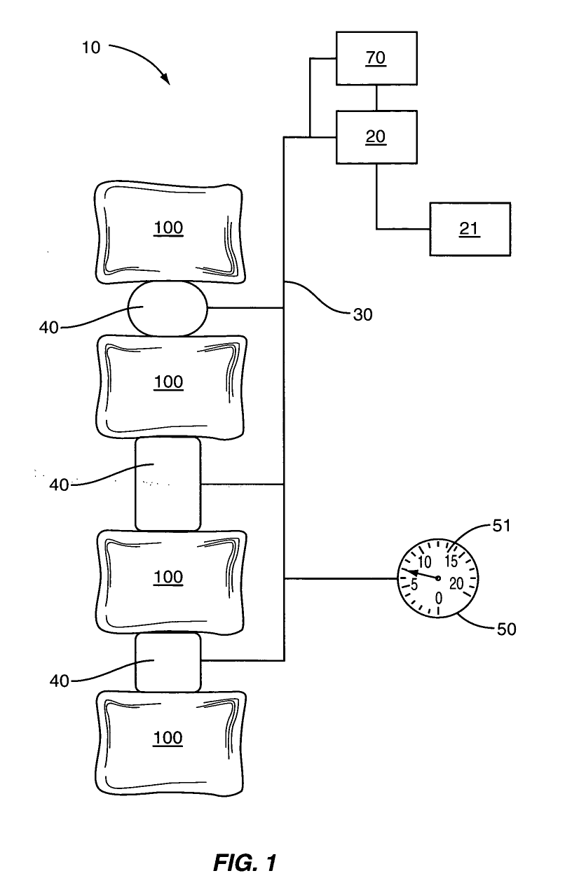

FIG. 1 illustrates one embodiment of a device generally illustrated as element 10 having a power source 20, supply line 30, and adjustable members 40. An adjustable member 40 is positioned between vertebral members 100 over two or more spinal levels. In this embodiment, the supply line 30 extends between each of the members 40 and the power source 20. Activation of the power source 20 may feed power through the supply line 30 and to the adjustable members 40. In one embodiment, members 40 move from a closed orientation towards an open orientation to space apart the vertebral members 100.

In one embodiment, power source 20 provides power to the members 40 to move from the closed orientation towards the open orientation. In one embodiment, the system uses a fluid to adjust the orientation of the members 40. In one specific embodiment, the system uses a hydraulic fluid. In one embodiment, a reservoir 21 may be operatively connected with the power source 20 for holding the fluid when it is not within the supply line 30 or members 40. Reservoir 21 may be an integral with or remotely located from the power source 20. In one embodiment, power source 20 includes a pump for moving the fluid through the supply line 30 and into each of the members 40. Power source 20 in one embodiment is adjustable to move fluid into the supply line 30 at various speeds and at various pressures as necessary for the necessary vertebral spacing. In one embodiment, power source 20 may further operate in a reverse direction to pull the fluid from the members 40. The reverse movement of the fluid from the members 40 towards the power source 20 may cause the members 40 to move from the open orientation towards the closed orientation.

Another embodiment includes a power source 20 that moves gas including air. In one embodiment, power source 20 is a compressor that moves the gas into the supply line 30 and members 40. Another embodiment features an electrical power source 20. In one embodiment, expandable members 40 are electrically actuated and movable between the open and closed orientations. Each member 40 may include a torque limiter to control the extent of force applied to the vertebral members 100.

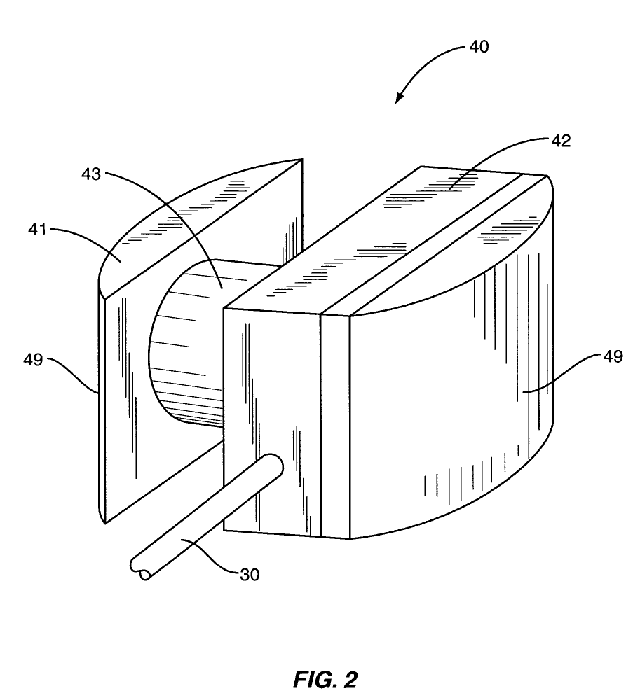

In one embodiment, members 40 are movable between open and closed orientations. In the embodiment, the members 40 are sized to fit within the intervertebral disc space formed between the vertebral members 100 when in a closed orientation. FIG. 2 illustrates one embodiment of a member 40 having a first section 41 and a second section 42. Contact surfaces 49 may be positioned on the outer edges of the sections 41, 42 to contact the vertebral members 100. In one embodiment, first section 41 includes an extension arm 43 that fits within the second section 42 in the closed orientation. In one embodiment, the extension arm 43 extends outward from the second section 42 in the open orientation to space apart the contact surfaces 49.

In one embodiment, contact surfaces 49 may be contoured and/or shaped to correspond to the geometry of the vertebral members 100. Further, contact surfaces 49 in one embodiment may be removably connected to the first and second sections 41, 42 and are replaceable as necessary to match the geometry of the vertebral members 100. Members 40 and the contact surfaces 49 may be shaped to simulate lordotic implants or include implant shaped endplates so the surgeon can template the final implant size in height, width, and depth.

In one embodiment, one or both sections 41, 42 include a connection for attachment of the supply line 30. Introduction of fluid, gas, or electricity (hereinafter called power) in one embodiment into the member 40 causes the sections 41, 42 to expand thereby increasing the height measured between the contact surfaces 49. In one embodiment, removal of the power from the member 40 causes the sections 41, 42 to move together thus decreasing the height. In one embodiment, member 40 includes a piston that actuates upon receipt of power through the supply line 30.

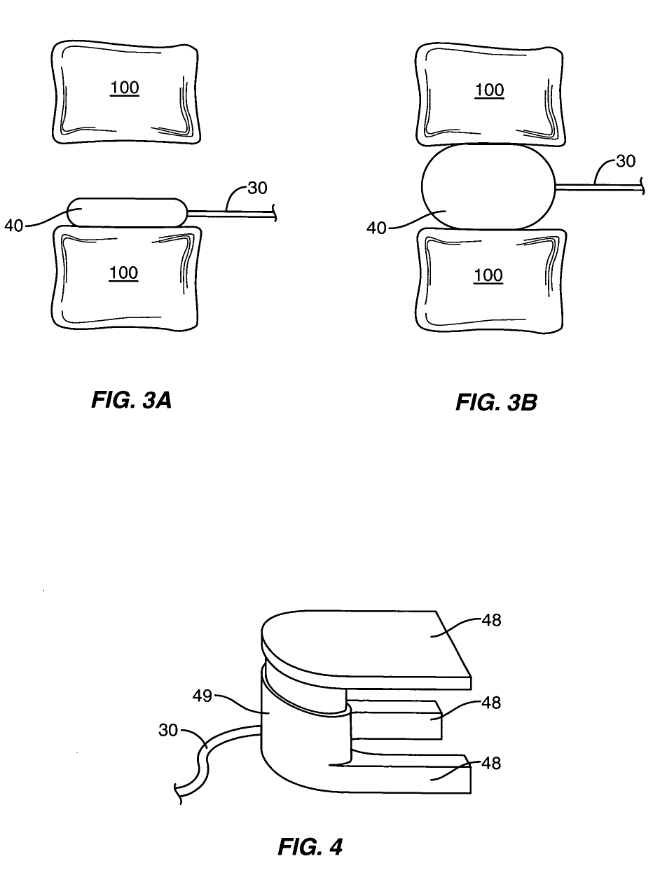

FIGS. 3A and 3B illustrate another embodiment of a member 40. In one embodiment, member 40 is of a unitary design having an enclosed interior that is operatively connected with the supply line 30. In one embodiment of the closed orientation as illustrated in FIG. 3A, member 40 includes a reduced height to fit within the intervertebral space between the vertebral members 100. In one embodiment, member 40 in the closed orientation is sized to contact only one vertebral member 100. In another embodiment, member 40 in the closed orientation may contact two or more vertebral members 100. In one embodiment of a member 40 in the open orientation as illustrated in FIG. 3B, member 40 includes a greater size. This increase in size causes the member 40 to contact both vertebral members 100 and apply a spacing force to the vertebral members 100. In one embodiment, member 40 may comprise an expandable or otherwise deformable material that expands when filled with gas or fluid such as water, saline solution, or the like.

FIG. 4 illustrates another embodiment of a member 40 having a body 49 and supports 48. In one embodiment, body 34 remains on the exterior of the intervertebral space formed between the vertebral members 100. Supports 48 extend into the intervertebral space and contact the vertebral members 100. In one embodiment as illustrated by the bottom supports 48 of FIG. 4, supports 48 include a limited width and are spaced apart forming a working region therebetween to allow for access to the surfaces of the vertebral members 100. The distance between the supports 48 and size of the working region may vary depending upon the context. In one embodiment as illustrated by the upper support 48 of FIG. 4, support 48 covers substantially the entirety of the surface of the vertebral member 100.

In one embodiment, different types of members 40 may be used at different spinal levels to space apart the vertebral members 100. In one embodiment as illustrated in FIG. 1, two or more different types of members are positioned within the space between the vertebral members.

In one embodiment, members 40 include a locking mechanism to lock the member 40 at a specific height. Locking members in one embodiment may maintain the height even after the power is removed from the member 40. In one method, member 40 is expanded to a height and a locking mechanism is activated to prevent further size changes. After activation, power source 20 can be deactivated without affecting the height of the locked member 40. In one embodiment, the locking mechanism is a valve for maintaining fluid pressure within the member 40. In another embodiment, locking mechanism is a hermetic seal for maintaining gas pressure within the member 40. In another embodiment, locking mechanism is an electronic circuit for maintaining a current or voltage to the member 40.

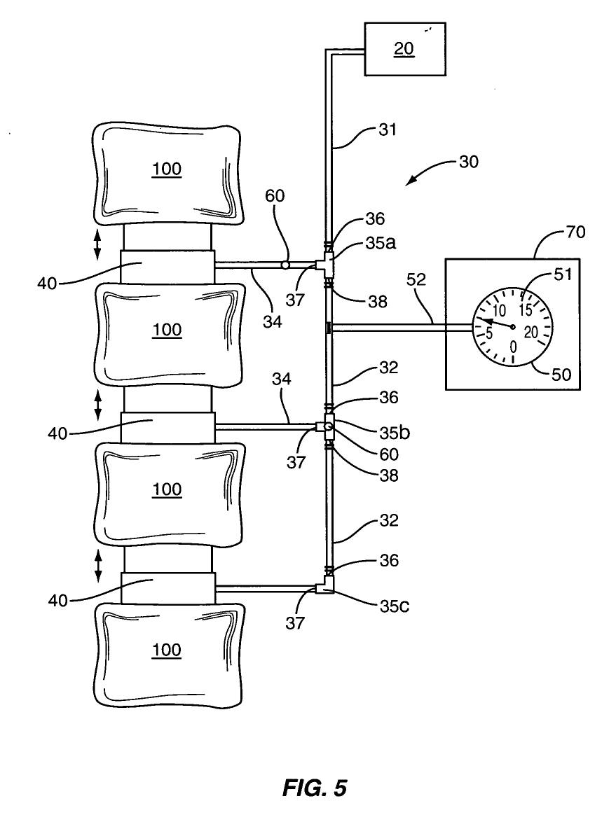

Supply line 30 moves fluid between the power source 20 and the members 40. The supply line 30 may include the same size between the power source 20 and the members 40, or may include different sizes. In one embodiment, more than one supply line 30 extends between the power source 20 and one or more of the members 40. In one embodiment as illustrated in FIG. 5, supply line 30 includes a main line 31 that extends between the power source 20 and a first connector 35a. A secondary line 32 connects to the downstream side of the first connector 35a and extends to a second connector 35b, and eventually to a third connector 35c. Feed lines 34 extend from each of the connectors 35 to a member 40. In one embodiment, main line 31 may include a larger size than either of secondary 32 and feed lines 34 because it may be required to handle a larger capacity of power than the other two lines.

In one embodiment, connectors, generally referred to as 35, connect together the various lines of the supply line 30. One connector type 35a, 35b, includes a three-way connection having a first and second connections 36, 38 along a first section of the supply line 30, and a third connection 37 that connects with the feed line 34 that leads to and from the member 40. A second connector type 35c includes first and third connections 36, 37 as described above. In another embodiment (not illustrated), the farthest secondary line 32 from the power source 20 connects directly with one of the members 40.

In one embodiment, one or more valves 60 may be positioned along the supply line 30 to control the power leading into the members 40. In one embodiment, each of the valves 60 independently control the power introduced into each one or more members 40. In one embodiment, valves 60 may be selectively positionable between open and closed orientations. In one embodiment of the open orientation, the amount of power fed out of the valve 60 is the same that is fed further downstream along the supply line without any affect. In one embodiment of the closed orientation, the amount of power fed from the valve 60 is less than the power fed into the valve 60. In one embodiment, valve 60 can control the amount of power feed from about 100% (i.e., in an open orientation) to about 0% (in a closed orientation).

Valves 60 may be positioned at a variety of locations along the supply line 30. In one embodiment as illustrated in FIG. 5, a valve 60 is positioned along the feed line 34 extending between connector 35a and member 40. In one embodiment, more than one valve 60 may be placed along a section of the supply line 30. In one embodiment, multiple valves 60 create safety measures in the event of failure of the power source 20 or other valve 60 along the same supply line 30. In one embodiment as illustrated in FIG. 5, a valve 60 is mounted within the connector 35b.

In one embodiment, an indicator 50 may be operatively connected to the supply line 30 to detect the amount of power within the supply line 30. In one embodiment, indicator 50 includes a gauge 51 for visual observation of the power. In one embodiment as illustrated in FIG. 5, indicator 50 is connected with the supply line 30 through a line 52. Indicator 50 may be positioned at a variety of locations along the supply line 30. In one embodiment as illustrated in FIG. 5, indicator 50 is positioned between the power source 20 and end of the supply line 30. In another embodiment, indicator 50 is positioned at the furthest point from the power source 20. In one embodiment, indicator 50 may be directly connected with the power source 20. In one embodiment, more than one indicator 50 may be connected along the supply line 30.

In one embodiment, a feedback system 70 may be operatively connected with the device 10 to provide immediate, real-time, and/or requested information to the surgeon regarding one or more of the device characteristics. Feedback system 70 may be independent or associated with the indicator 50. In one embodiment, feedback system 70 provides an indication when a desired or predetermined separation characteristic of the members 40 is obtained, and/or when certain threshold separation characteristics are obtained and/or approached. By way of example, system 70 can provide the force being exerted by each of the members 40 to the vertebral members 100, and the resulting spacing of the vertebral members 100.

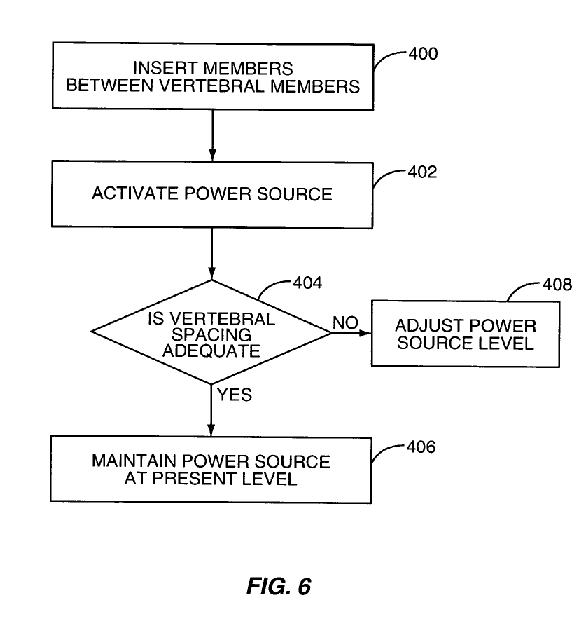

FIG. 6 illustrates the steps of one method of spacing vertebral members. In one embodiment, the desired vertebral spacing is determined prior to insertion of the members 40. In one embodiment, the spacing is determined through pre-operative planning or anatomical studies. In one embodiment, the spacing may correspond to a maximum pressure or tension that is to be applied to the vertebral members 100. Once the spacing is determined, an incision is made to access a surgical site on or near the spinal column. The members 40 are inserted in the incision and placed relative to the vertebral members 100 (step 400). In one embodiment, members 40 are placed within the patient in intervertebral spaces between the adjacent vertebral members 100, with the power source 20 being positioned exterior to the patient.

Once each member 40 is inserted, in one embodiment the power source 20 is activated to supply power into the supply line 30 (step 402). The fluid moves through the supply line and into each member 40 thereby causing the member height to increase. In one embodiment, a substantially equal amount of power is introduced into each member 40 thus causing each member to apply the same force to the vertebral members 100. In one embodiment, the applied force is substantially the same, regardless of the starting size of the intervertebral disc space or final distraction magnitude. By way of example using FIG. 1, a force applied through a first member (i.e., the top-most member as viewed in FIG. 1) causes the vertebral members to distract a first amount. The same force applied through a second member causes the vertebral members to distract a different amount.

In one embodiment, at some point in the process, the spacing between the vertebral members 100 is measured (step 404). In one embodiment, physical measurements of the vertebral member spacing are taken periodically during the process. If additional spacing is required, the power source 20 is adjusted accordingly (step 408). If the spacing is adequate, the expansion process is complete (step 406).

In one embodiment, once spacing is adequate replacement spacers are inserted and take the place of the members 40. In one embodiment, removal of the members 40 includes operating the power source 20 in a second direction and drawing power from each member 40 causing the height to decrease to an amount that the members can be removed. In one embodiment, the heights of each of the members 40 decreases at the same amount as power is equally drawn from each member 40. In one embodiment, each member 40 is independently moved towards the closed orientation.

In one embodiment, valves 60 act as the locking mechanisms to control the size of the members 40. Turning the valve 60 from an open to a closed position while in the open orientation prevents a reduction in the member size.

One embodiment includes accessing the spine from an anterior approach to the cervical spine. Other applications contemplate other approaches, including posterior, postero-lateral, antero-lateral and lateral approaches to the spine, and accessing other regions of the spine, including the cervical, thoracic, lumbar and/or sacral portions of the spine.

The embodiments described above feature the members 40 positioned within the intervertebral space formed between adjacent vertebral members. The members 40 may also be used for spacing other sections of the spine, including pedicles, lamina, and processes.

In one embodiment, a single member 40 is positioned between the vertebral members 100. In one embodiment, multiple members 40 are positioned between the same vertebral members 100 to work in combination to achieve the proper spacing.

In one embodiment, the device is modular in the sense that additional members 40 may be added and deleted from the supply line 30. By way of example, the device illustrated in FIG. 5 may be increased to add another member 40. This may be accomplished by replacing connector 35c with a three-way connector, such as 35a, and adding additional length to the supply line that extends to another member 40. Likewise, the device 10 may be decreased in size. Again using the example of FIG. 5, connector 35b can be replaced with a two-way connector such as 35c to form a device having two members 40.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting.

The present invention may be carried out in other specific ways than those herein set forth without departing from the scope and essential characteristics of the invention. More than one power source 20 may be attached to the supply line 30. In one embodiment, members 40 remain within the patient in an open orientation during additional surgical procedures. In one embodiment, drawing the power from the member 40 comprises deactivating the power source 20. The present embodiments are, therefore, to be considered in all respects as illustrative and not restrictive, and all changes coming within the meaning and equivalency range of the appended claims are intended to be embraced therein.

What is claimed is:

1. A device to space vertebral members comprising:
first and second expandable members;
a power source; and
a supply line operatively connecting the power source with each of the first and second members;
wherein the power supply is adapted to feed power through the supply line and to each of the first and second expandable members causing the members to increase in size and apply a force to the vertebral members, the force applied by the first and second expandable members being substantially equal.

2. The device of claim 1, wherein the power source is adapted to supply a pressurized material through the supply line to each of the first and second members.

3. The device of claim 2, wherein the pressurized material comprises hydraulic fluid that is moved by the power source through the supply line and into each of the first and second members.

4. The device of claim 2, wherein the pressurized material comprises a gas that is moved by the power source through the supply line and into each of the first and second members.

5. The device of claim 1, wherein the power source is adapted to supply electricity through the supply line to the first and second members.

6. The device of claim 1, further comprising a connector positioned along the supply line, the connector including a first connection with the supply line and a second connection with a feed line that leads to one of the members.

7. The device of claim 6, wherein the connector further comprises a stop at a remote point along the supply line away from the power source.

8. The device of claim 1, wherein the first and second members each include a different construction.

9. A device to space vertebral members along the spine, the device comprising:
a first member positioned within a first space along the spine;
a second member positioned within a second space along the spine;
a power source; and
a supply line operatively connecting the power source with each of the first and second members;
wherein the power source is adapted to supply power through the supply line to each of the first and second members, the power causing the first and second members to exert a force to the vertebral members adjacent to the first and second spaces, the force applied by each of the first and second members being substantially equal.

10. The device of claim 9, wherein the supply line and power source are adapted to remove the power from the first and second members to decrease the force applied to the vertebral members.

11. The device of claim 9, wherein the supply line comprises a main line feeding outward from the power source and a feed line that extends between the main line and the first member.

12. The device of claim 11, further comprising a connector at the intersection of the main line and the feed line, the connector having a first connection to connect with the main line and a second connection to connect with the feed line.

13. The device of claim 11, wherein the connector further comprises a third connection to connect to a secondary line that leads to the second member.

14. The device of claim 9, further comprising an indicator placed along the supply line to determine a pressure of the material within the supply line.

15. A device to space vertebral members along the spine, the device comprising:
first and second members positioned within spaces along the spine, the members being positionable between a first orientation having a first size and a second orientation having a second greater size;
a power source to move the first and second members between the first and second orientations; and
a supply grid operatively connecting the power source with the first and second members;
the first and second members movable between the first and second orientations dependent upon power supplied from the power source, the supply grid causing the power to be substantially equal within the first and second members while moving between the first and second orientations and causing the first and second members to each exert a force to the vertebral members that is substantially equal.

16. The device of claim 15, wherein the power source is adapted to move a hydraulic fluid through the supply grid and the first and second members.

17. The device of claim 16, further comprising a reservoir for holding the hydraulic fluid that is outside of the supply grid and the first and second members.

18. The device of claim 15, wherein the power source is adapted to move a gas through the supply grid and the first and second members.

19. The device of claim 15, wherein the first and second members are electrically powered and the power source is adapted to supply electricity through the supply grid to each of the first and second members.

20. A method of spacing vertebral members comprising the steps of:
inserting a first member within a first intervertebral space;
inserting a second member within a second intervertebral space;
supplying power to each of the first and second members and causing the members to expand in size; and
expanding the first and second intervertebral spaces by applying a first force through the first member to the first intervertebral space that is substantially equal to a second force that is applied through the second member to the second intervertebral space; and
maintaining the first and second forces substantially equal during the expanding of the first and second intervertebral spaces.

21. The method of claim 20, wherein the step of supplying the power comprises using a common power source and supplying the power to each of the first and second members.

22. The method of claim 20, further comprising expanding the first and second members to different sizes while maintaining the first and second forces substantially equal.

23. The method of claim 20, wherein the step of supplying the power to each of the first and second members and causing the members to expand comprises supplying equal amounts of fluid to each of the first and second members.

24. A method of spacing vertebral members comprising the steps of:
inserting a first member at a first spinal level;
inserting a second member at a second spinal level;
activating a power supply and feeding power through a grid to the first and second members;
causing the first and second members to expand in size from a first size towards a second size; and
maintaining an equal distraction force while the first and second members expand from the first size towards the second size.

25. The method of claim 24, further comprising expanding the members to different sizes and maintaining the distraction force to be substantially equal over the first and second spinal levels.
